(19) United States
(12) Patent Application Publication
(10) Pub. No.: US 2023/0258639 A1
(43) Pub. Date: Aug. 17, 2023

(54) Assay to Differentiate Natural CMV Infection from CMV Vaccines that Lack UL144

(71) Applicant: The Medical College of Wisconsin, Inc., Milwaukee, WI (US)

(72) Inventor: Ravit Boger, Milwaukee, WI (US)

(21) Appl. No.: 17/965,148

(22) Filed: Oct. 13, 2022

Related U.S. Application Data
(60) Provisional application No. 63/255,308, filed on Oct. 13, 2021.

(51) Int. Cl. G01N 33/569; G01N 33/543

(52) U.S. Cl. CPC G01N 33/56994; G01N 33/54388; G01N 2469/20; G01N 2470/04; G01N 2333/045

(57) ABSTRACT

The present invention provides methods and immunoassays kits for the improved detection of cytomegalovirus (CMV) in a subject. In some embodiments, the methods and immunoassays kits differentiate between subjects who have developed a serological response to vaccination against CMV and subjects who have had a natural infection. In some embodiments, the methods and immunoassays kits differentiate between serological responses to different CMV serotypes.

### CROSS-REFERENCE TO RELATED APPLICATIONS

This application claims the benefit of U.S. provisional patent application No. 63/255,308, filed on Oct. 13, 2021, the provisional application is incorporated herein by reference in its entirety.

### STATEMENT REGARDING FEDERALLY SPONSORED RESEARCH

Not applicable.

### SEQUENCE LISTING

A Sequence Listing accompanies this application and is submitted as an ASCII text file of the sequence listing named “650053_00911.xml” which is 13,135 bytes in size and was created on Oct. 13, 2022. The sequence listing is electronically submitted via EFS-Web with the application and is incorporated herein by reference in its entirety.

### BACKGROUND

Infection with human cytomegalovirus (CMV) is common and may have grave consequences in transplant recipients and congenitally infected children. Diagnosis of CMV infection is based on detection of specific antibodies and molecular assays. The incorporation of CMV serological assays into diagnostic algorithms requires careful evaluation and interpretation. Very few serological assays measure CMV infection by a specific strain. Such assays may provide important tools for vaccine trials and epidemiological studies.

The advancement of molecular assays for monitoring cytomegalovirus (CMV) disease has improved clinical outcome, allowing for timely therapeutic decisions. However, establishing CMV serostatus in pregnant women and organ donors/recipients remains critical for further diagnostic and therapeutic considerations. Antibody measurements are particularly useful in determining risk of CMV acquisition in seronegative patients.

Determination of CMV acquisition or exposure in previously CMV seronegative individuals is based on interval testing. CMV DNAemia is detectable for a short time in plasma, although CMV DNA can be detected in urine and saliva for up to several months. An antibody-based assay may provide a practical and accurate assessment of CMV serostatus. The commercially available ELISA assays are based on detection of antibody responses to whole virus preparations prepared from attenuated laboratory adapted strains such as AD169. Recognizing the importance of CMV strain variation, several ELISA methods were developed to identify strain-specific antibodies against the CMV glycoproteins gH and gB. Furthermore, the development of CMV vaccines resulted in the need for sensitive and specific assays for follow up of infections after immunization. For that reason, concomitant with the development of CMV glycoprotein B vaccines, an IgG ELISA assay was developed using UL32 (pp150) as capture antigen, to eliminate the need for gB pre-absorption and verification.

Immunization with CMV vaccines based on the AD169 strain may elicit neutralizing antibody responses that are indistinguishable from neutralizing antibodies to natural infection.

Accordingly, there remains a need in the art for better immunoassays and methods for distinguishing not only individuals that had a natural infection or were vaccinated, but other methods of determining the CMV serotype the individual was infected with.

### SUMMARY

In an aspect of the current disclosure, immunoassays for detecting cytomegalovirus in a biological sample are provided. In some embodiments, the immunoassays comprise one or more of the following: (a) a capture reagent comprising a purified, recombinant polypeptide fragment of UL144 protein, and (b) a detection reagent. In some embodiments, the capture reagent comprises a UL144 protein sequence comprising a protein sequence selected from SEQ ID NO: 4-6 or a sequence having at least about 90% sequence similarity to SEQ ID NO:4-6. In some embodiments, the capture reagent is attached to a solid or semi-solid support. In some embodiments, the capture reagent is immobilized on the solid or semisolid support. In some embodiments, the capture reagent is coated on a microtiter plate. In some embodiments, the detection agent is a detectable antibody. In some embodiments, the antibody is an anti-IgG antibody. In some embodiments, the detectable antibody is a monoclonal antibody. In some embodiments, the detectable agent is biotinylated and the detection means is avidin or streptavidin-peroxidase and 3,3′,5,5′-tetramethyl benzidine. In some embodiments, the detection is colorimetric and the immunoassay further comprises reagents for colorimetric detection. In some embodiments, the detectable agent is amplified by a fluorometric reagent. In some embodiments, the immunoassay is a multiplex assay capable of detecting two or more CMV serotypes, the immunoassay comprising: a) a first capture reagent to a first serotype and a second capture reagent to a second serotype, wherein the two capture reagents are in different detection zones in the assay; and b) the detection agent, wherein the two different detection zones are able to detect the two or more CMV serotypes. In some embodiments, the assay further comprises a third capture reagent to a third serotype, wherein the third capture reagent is in a third detection zone. In some embodiments, the first, second and third capture reagent are selected from i) UL144A protein of serotype A or a polypeptide having at least 90% sequence similarity to UL144A serotype A; ii) UL144A protein of serotype B or a polypeptide having at least 90% sequence similarity to UL144A serotype B; and iii) UL144A protein of serotype C or a polypeptide having at least 90% sequence similarity to UL144A serotype C. In some embodiments, the detection zones may be separate wells on a microtiter plate. In some embodiments, the detection zones may be separate channels in a lateral flow device. In some embodiments, the first, second and third capture reagents are selected from i) UL144A protein of serotype A of SEQ ID NO:4 or a polypeptide having at least 90% sequence similarity to UL144A serotype A; ii) UL144A protein of serotype B of SEQ ID NO:5 or a polypeptide having at least 90% sequence similarity to UL144A serotype B; and iii) UL144A protein of serotype C of SEQ ID NO:6 or a polypeptide having at least 90% sequence similarity to UL144A serotype C.

In an aspect of the current disclosure, methods of detecting cytomegalovirus infection in a subject are provided. In some embodiments, the method comprising: (a) providing an immunoassay comprising one or more capture reagents specific for CMV serotype A, B or C, preferably wherein the capture reagent is bound to a solid or semi-solid support; (b) contacting the immunoassay with a biological sample under conditions which allow of CMV antibodies if present in the biological sample to bind to the one or more capture reagents; (c) adding the detection reagent, and (d) detecting the complex formed between the capture reagent, CMV antibody and detection reagent within the immunoassay. In some embodiments, the one or more capture reagents are selected from i) UL144A protein of serotype A of SEQ ID NO:4 or a polypeptide having at least 90% sequence similarity to UL144A serotype A; ii) UL144A protein of serotype B of SEQ ID NO:5 or a polypeptide having at least 90% sequence similarity to UL144A serotype B; and iii) UL144A protein of serotype C of SEQ ID NO:6 or a polypeptide having at least 90% sequence similarity to UL144A serotype C. In some embodiments, the method differentiates between subjects who have been infected with CMV and subjects who have been vaccinated against CMV.

In an aspect of the current disclosure, methods for detecting the presence of CMV antibodies in a test sample are provided. In some embodiments, the methods comprising the steps of: a) providing a test sample suspected of containing CMV antibodies; b) adding a quantity of the polypeptide selected from SEQ ID NO:4-6 to the sample, the quantity being sufficient to produce a detectable level of binding activity by CMV antibodies in the test sample; and c) detecting the presence of CMV bound to said polypeptide in the test sample by a detection reagent.

### BRIEF DESCRIPTION OF THE DRAWINGS

FIG. 1: Background noise reduction and seroreactivity to UL144 B. A) CMV positive and negative sera were diluted 1:40 in blocking buffer and tested for absorbance to 96-well plates in the absence of coating antigen. All steps performed were identical to the protocol for the UL144 ELISA, but with an overnight incubation in PBS without coating antigen. B) De-identified serum samples were diluted 1:40, 1:80 or 1:160 in blocking buffer and tested for reactivity to UL144 B. One well was tested for each serum sample per dilution. The average value of the negative samples plus 3×SD of the negative samples was used to determine a cutoff OD. The OD value of the serum sample minus the cutoff OD was calculated and plotted on the graph.

FIG. 2: Seroreactivity to UL144 A, B, or C. Clinical ELISA positive serum samples were tested in duplicate for reactivity to UL144 A, UL144 B, or UL144 C. For each antigen, three clinical ELISA negative serum samples were used to determine the cutoff, and OD values were calculated as described in FIG. 1.

FIG. 3: Initial multiplex assay and optimization coating antigen conditions. A) Serum samples were tested using multiplexed antigen coating conditions of 1.25 μg/ml UL144 A, 0.625 g/ml UL144 B and 0.3125 μg/ml UL144 C, and the OD values were calculated. B) Three serum samples were tested against different combinations of antigen concentrations: high positive, low positive and negative for CMV reactivity. The average OD values of duplicate wells for a given multiplexed condition was calculated, and the positive serum OD value was divided by the negative serum OD value to determine the binding ratio (left). Combination of tested concentrations of UL144 A, B, or C in the multiplex assay are provided on the right.

FIG. 4: Threshold determination, ROC curve, and validation of multiplex UL144 ELISA.

A) 144 serum samples, 55 positive and 59 negative by the clinical ELISA, were tested for reactivity to UL144. The dashed line represents the cutoff value of 0.1. B) ROC curve was generated from data of 4A. C) Serum samples were tested for reactivity to UL144. The dashed line represents the threshold of 0.1 as determined by the ROC curve in FIG. 4A.

FIG. 5: Interassay agreement of blinded samples and long-term freezing of antigen-coated plates. A) Twenty serum samples were tested for reactivity to UL144 on five separate days. The dashed line represents the threshold (0.1) from the ROC curve as described in FIG. 4A. Each color represents the OD value at a specific time of testing. B) Plates were coated with the antigen cocktail overnight, washed, and gently patted dry prior to freezing at −80° C. Serum reactivity was tested without freezing, or after 1, 2, 4 or 8 weeks frozen.

FIG. 6. Amino acid sequences of UL144A, B, and C and proteins used in the ELISA development.

FIG. 7. Provides an overall design scheme of one embodiment of the present invention.

FIG. 8. Binding of UL144 B to 96-well plates. Decreasing concentrations of UL144 B were plated on Immulon IIB or Maxisorp (44-2404, Invitrogen, Carlsbad, Calif.) 96 well plates and incubated overnight. The plates were blocked for one hour, followed by detection of UL144 B using 0.5 μg/ml HRP-tagged anti-6×His Tag Antibody (A00612, GenScript) diluted in blocking buffer. The plates were developed as described in the Methods section. Shown are average values of single wells from two experiments.

FIG. 9. Amino acid sequence analysis of a UL144 B (F50), UL144 A B (F5 and F31) and UL144 C (F30) positive serum compared to known UL144 A, UL144 B and UL144 C amino acid sequences. UL144 PCR was performed from CMV positive DNA sera followed by Sanger sequencing. UL144 nucleotide sequence was translated to amino acid sequence, and an alignment with UL144 A, B and C was performed using Clustal Omega. UL144A sequence is SEQ ID NO: 1, UL144B is SEQ ID NO: 2, UL144C is SEQ ID NO: 3, F50 is SEQ ID NO: 10, F5 is SEQ ID NO: 11, F31 is SEQ ID NO: 3.

FIG. 10: Cross-reactivity of HSV1 positive sera to UL144. Twelve deidentified, CMV seronegative, HSV1 seropositive samples were tested for reactivity to the UL144 ELISA. The dashed line represents the threshold value (0.1) from the ROC curve as described in FIG. 4a.

FIG. 11: Is the UL144A amino acid sequence (SEQ ID NO: 4) and a schematic of the codon optimized nucleic acid sequence for UL144A (SEQ ID NO: 7).

FIG. 12: Is the UL144B amino acid sequence (SEQ ID NO: 5) and a schematic of the codon optimized nucleic acid sequence for UL144A (SEQ ID NO: 8).

FIG. 13: Is the UL144C amino acid sequence (SEQ ID NO: 6) and a schematic of the codon optimized nucleic acid sequence for UL144A (SEQ ID NO: 9).

### DETAILED DESCRIPTION

The present invention provides an immunoassay, particularly a multiplex immunoassay, methods and kits for detecting cytomegalovirus antibodies in a subject, particularly the ability to distinguish between A, B and C serotypes and also the ability to distinguish a subject who has mounted an antibody response to natural CMV infection as opposed to a person who has been immunized against CMV.

To address limitations of currently available immunoassays which cannot distinguish antibodies produced from a natural infection versus immunized individuals, the inventor developed a new multiplex ELISA assay for CMV protein UL144. The UL144 gene, present in the UL/b′ boundary of the CMV genome, is present in clinical isolates but absent in AD169 and other laboratory-adapted strains. The inventor defined three major, distinct UL144 genotypes from multiple original samples and clinical isolates. The three major UL144 genotypes (A, B, and C) and recombinants have similar distribution in different geographical locations (9, 12, 13), and specific genotypes may be associated with severe sequelae of congenital CMV infection (8, 12).

UL144 is a membrane-anchored glycoprotein that is largely intracellular, but it is also found on the cell surface when overexpressed (14, 15). The inventor characterized an ELISA method for measurement of the serum antibody response to the three UL144 types as demonstrated in the Examples.

The immunosorbent assay, which may be an enzyme-linked immunosorbent assay (ELISA) using CMV-encoded UL144 as antigen or capture reagent. Genetic analysis of UL144 identified three major genotypes, A, B and C, and recombinants. The ELISA was developed with the three UL144 proteins and optimized as a multiplex assay. Sera from 55 positive and 59 negative CMV IgG, determined by the clinical microbiology laboratory, were used for evaluation and optimization. A cutoff optical density (OD) that distinguishes UL144 antibody-positive from -negative sera was established. A panel of 75 sera detected UL144 A, B, C and combination of these antigens. Assay threshold of 0.1 OD was determined using 114 sera, followed by validation using 189 sera. The overall sensitivity, specificity, positive and negative predictive values of the multiplex ELISA were 86.72% (95% CI 79.59% to 92.07%), 96.57% (92.69% to 98.73%), 94.40% (88.45% to 97.38%), and 91.60% (87.50% to 94.44%). The inter- and intra-assay median coefficients of variation were 0.06 (IQR 0.56, 0.2) and 0.171 (IQR 0.038, 0.302). This ELISA gives simple and reproducible results for detection of anti-CMV UL144 IgG. It may assist in differentiating natural infection from CMV vaccines that lack UL144.

Assays

In an aspect of the present disclosure, the present invention provides an immunoassay, or immunoassay kits, for detecting cytomegalovirus in a biological sample. In some embodiments, the immunoassay comprises one or more of the following: (a) a capture reagent comprising a purified, recombinant polypeptide of UL144 protein or fragment thereof, and (b) a detection reagent. In some embodiments, the capture reagent is a UL144 peptide encoded by the genetically modified, codon optimized nucleic acid sequence shown in FIG. S6-S8).

As used herein, “cytomegalovirus” or “CMV” refers to human betaherpesvirus 5 (HHV-5). The primary host of HHV-5 is humans or Homo sapiens.

As used herein, “UL144” is a protein also known as “Membrane glycoprotein UL144”. In some embodiments UL144 has a sequence of SEQ ID NO: 3, or a sequence having at least 90% similarity thereto. In some embodiments UL144 has a sequence of SEQ ID NO: 4, or a sequence having at least 90% similarity thereto. In some embodiments UL144 has a sequence of SEQ ID NO: 5, or a sequence having at least 90% similarity thereto. SEQ ID NOs: 3-6 correspond to serotypes A, B, and C, respectively.

As used herein, “immunoassays” are assays or tests that in some fashion use the binding of antibodies to antigens to identify and detect certain particles. In some embodiments, the immunoassays of the present disclosure detect the exposure of a subject to the CMV antigens, and specifically to the CMV protein UL144. In some embodiments, UL144 has distinct sequences that relate to the particular strain from which the UL144 is derived, for example, UL144A protein of serotype A of SEQ ID NO:1 or a polypeptide having at least 90% sequence similarity to UL144A serotype A, UL144A protein of serotype B of SEQ ID NO:2 or a polypeptide having at least 90% sequence similarity to UL144A serotype B, and UL144A protein of serotype C of SEQ ID NO:3 or a polypeptide having at least 90% sequence similarity to UL144A serotype C. In some embodiments, the immunoassays of the present disclosure detect antibodies directed against UL144.

The immunoassays of the present disclosure provides one or more capture agents specific to CMV UL144 protein or fragment thereof, and a detection agent. The CMV UL144 protein or fragments thereof are produced recombinantly, e.g., using plasmids and vectors and tissue culture cells and include non-naturally occurring sequences, e.g., a tag, that allows for the purification of the recombinant protein from cellular proteins. Further, the plasmid or nucleic acid sequence used to recombinantly produce the protein in vitro has been codon optimized and therefore the protein described herein are not derived from a natural sequence. The non-naturally occurring codon optimized nucleic acid sequence that encodes UL144A, B and C can be found in FIG. S6-S8, respectively.

The term “capture agent” refers to a molecule or compound capable of binding a molecule of interest. In the present invention, the capture reagent is the CMV UL144 protein or fragments thereof that are capable of specific binding to antigens. In some embodiments, the immunoassay comprises two or more capture agents, each capture agent to a different serotype of CMV. In another embodiment, the immunoassay comprises three capture agents, e.g., a CMV serotype A UL144 protein or fragment thereof, a CMV serotype B UL144 protein or fragment thereof and a CMV serotype C UL144 protein or fragment thereof. Specifically, the CMV UL144 proteins used in the immunoassays described herein are made from codon-optimized recombinant proteins produced in vitro using molecular cloning techniques and are not naturally occurring. Further, in some embodiments, the capture agent comprises a tag that allows it to be (a) purified, (b) bound to a solid or semi-solid surface, or c) both purified from cells in culture and bound to a solid or semi-solid surface. Suitable tags are known in the art and are described herein.

In some embodiments, capture reagents are linked to a solid support or a semi-solid support. In some embodiments, capture reagents are recombinant CMV UL144 protein coated to a surface of a solid support or a semi-solid support.

Specifically, the immunoassay can comprise a capture reagent comprises a UL144 protein sequence comprising a protein sequence selected from SEQ ID NO: 4-6 or a sequence having at least 90% sequence similarity to SEQ ID NOs: 4-6, alternatively a sequence having at least 95% sequence similarity to any one of SEQ ID NOs: 4-6, alternatively a sequence having at least 98% sequence similarity to any one of SEQ ID NOs: 4-6, alternatively a sequence having at least 99% sequence similarity to any one of SEQ ID NOs: 4-6. In some embodiments, the amino acid sequences SEQ ID NOs: 4-6 are encoded by the codon optimized DNA sequences SEQ ID NOs: 7-9, respectively. In some embodiments the amino acid sequences SEQ ID NOs: 4-6 are encoded by DNA sequences with at least 90%, at least 95%, at least 96%, at least 97%, at least 98%, or at least 99% similarity to SEQ ID NOs: 7-9.

As used herein, “solid support” refers to a solid-phase material that is capable of acting as a support. In some embodiments, exemplary solid supports include microplates, microtiter plates, glass slides, microfluidic devices, and lateral flow devices. Solid supports may comprise distinct zones in which each zone is separated by distance and/or a physical barrier such that more than one capture agent can be used simultaneously. In some embodiments, the lateral flow devices comprise separate channels.

As used herein, “semi-solid support” refers to a semi-solid phase material that is capable of acting as a support. In some embodiments, exemplary semi-solid supports include, but are not limited to, gel, filters, or hydrogel beads.

As used herein, “detection reagents” are reagents used to signal a particular binding outcome in the disclosed immunoassays. In some embodiments, exemplary detection reagents are a protein or molecules that can specifically bind to human antibodies, particularly, that can bind to human immunoglobulin G protein, and in some embodiments, to the constant region of the antibodies such that it can specifically bind antibodies from a human subject. In some embodiments, the detection reagent is an antibody, for example, a anti-human immunoglobulin antibodies. The detection agent may be a protein or molecule further linked to a detectable marker. In some embodiments, exemplary detection reagents include anti-human IgG antibodies linked to a detectable marker.

As used herein, “detectable markers” or “detectable agents” refers to a variety of reagents known in the art that produce a visible signal, e.g., fluorescent molecules or fluorometric reagents, colorimetric reagents (otherwise known as reagents for colorimetric detection), or reagents that catalyze an enzymatic reaction that is visible, e.g., alkaline phosphatase, horseradish peroxidase, etc. In some embodiments, detectable markers are streptavidin linked to peroxidase. As used herein, “fluorometric reagents” are reagents that comprise a fluorescent molecule including, for example, fluorescein, green fluorescent protein (GFP), red fluorescent protein (RFP), alexafluor reagents, quantum dots, or other fluorescent molecules known in the art. In some embodiments, the presence of detection reagents are linked to biotin and are contacted with streptavidin linked to peroxidase, wherein visualization of the detection reagent is performed by contacting the detection reagent-biotin-streptavidin-peroxidase complex with tetramethyl benzidine (TMB) and incubating for a sufficient time to detect the detection reagent.

As used herein, “detection buffer” refers to any suitable buffer for visualizing a detection reagent. In some embodiments, the detection buffer is a phosphate buffered solution, a tris buffered solution, or other buffers known in the art.

In some embodiments, the present disclosure provides a multiplex immunoassay. The term “multiplex” refers to the ability of the assay to test for two or more capture reagents at the same time. Thus, the immunoassay is a multiplex assay capable of detecting two or more CMV serotypes. The multiplex immunoassay describe herein can comprise a) a first capture reagent to a first serotype and a second capture reagent to a second serotype, wherein the two capture reagents are assayed in different detection areas; and b) the detection agent, wherein the two different detection zones are able to detect the two or more CMV serotypes. In a preferred embodiment, the multiplex assay further comprises a third capture reagent to a third serotype, wherein the third capture reagent is in a third detection zone.

In some embodiments, the disclosure comprises a multiplex immunoassay comprising at least a first and a second capture agent. The first and the second capture agent comprise different capture agents to different CMV serotypes of UL144, wherein the UL144 proteins are selected from i) UL144A protein of serotype A or a polypeptide having at least 90% sequence similarity to UL144A serotype A; ii) UL144A protein of serotype B or a polypeptide having at least 90% sequence similarity to UL144A serotype B; and iii) UL144A protein of serotype C or a polypeptide having at least 90% sequence similarity to UL144A serotype C, and a detection agent. A polypeptide “having at least 90% sequence similarity” refers to a polypeptide having 90%, 91%, 92%, 93%, 94%, 95%, 96,%, 97%, 98%, 99% or 100% similarity to the reference polypeptide. The multiplex assay is capable of detecting both the first and second capture agent in the same assay. In some embodiments, the first and second capture agent are in the same assay but in separate detection regions or zones. In some embodiments, the detection zones may be separate regions, separate tubes, separate wells, or separate channels.

In some embodiments, the disclosure comprises a multiplex immunoassay comprises a first, a second and a third capture agent. In some embodiments, the first, second and third capture reagents are selected from i) UL144A protein of serotype A of SEQ ID NO.4 or a polypeptide having at least 90% sequence similarity to UL144A serotype A; ii) UL144A protein of serotype B of SEQ ID NO:5 or a polypeptide having at least 90% sequence similarity to UL144A serotype B; and iii) UL144A protein of serotype C of SEQ ID NO:6 or a polypeptide having at least 90% sequence similarity to UL144A serotype C. In some embodiments, the protein sequence has 92% sequence similarity, 95% sequence similarity, 98% sequence similarity, 99% sequence similarity to the one or more sequences for the serotype.

In some embodiments, the UL144A/B/C protein is encoded in a codon-optimized recombinant nucleic acid sequence. The nucleic acid sequence can be comprised within a vector, and the vector can be used to express the protein within cells in culture. In some embodiments, the codon-optimized recombinant nucleic acid sequences comprise SEQ ID NOs: 7-9, which encode UL144A, UL144B, and UL144C, respectively. In some embodiments, the recombinant nucleic acid sequences comprise sequences with at least 90%, at least 95%, at least 96%, at least 97%, at least 98%, or at least 99% similarity to SEQ ID NOs: 7-9.

As used herein, the term “construct”, “nucleic acid construct” or “DNA construct” refers to an artificially constructed (i.e., not naturally occurring) DNA molecule that is capable of expressing the polypeptide. Nucleic acid constructs may be part of a vector that is used, for example, to transform a cell. The term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”. Vectors suitable for use with the present invention comprise the constructs described herein and heterogeneous sequence necessary for proper propagation of the vector and expression of the encoded polypeptide. Preferably, the constructs are packaged in a vector suitable for delivery into a mammalian cell including but not limited to, an adeno-associated viral (AAV) vector, a lentiviral vector, or a vector suitable for transient transfection. As used herein, the term “vector,” “virus vector,” “delivery vector” (and similar terms) generally refers to a virus particle that functions as a nucleic acid delivery vehicle, and which comprises the viral nucleic acid (i.e., the vector genome) packaged within the virion. Suitable vectors are known and commercially available in the art. A skilled artisan will be familiar with the elements and configurations necessary for vector construction to encode the constructs described herein.

In some embodiments, the peptide or plurality of peptides used with the present invention further comprise a tag, preferably an exogenous tag or agent. The term “tag” or “agent,” as used herein, includes any useful moiety that allows for the purification, identification, or detection of the peptide(s) of the present invention. Any tag or agent that does not interfere with the ability of the peptide or peptides to bind to the antibodies within the sample may be used with the present invention. Suitable tags are known in the art and include, but are not limited to, affinity or epitope tags (e.g., cMyc, HIS, FLAG, V5-tag, HA-tag, NE-tag, S-tag, Ty tag, universal molecular identifier, magnetic beads, etc.) and florescent tags (e.g., RFP, GFP, etc.). Epitope tags are commonly used as a “purification tag”, i.e., a tag that facilitates isolation of the polypeptide from other non-specific proteins and peptides. For instance, the inventor included a 6×His tag in the peptides to allow them to be purified by nickel affinity chromatography using standard methods known in the art. In some embodiments, the epitope peptide and the tag are encoded in one nucleic acid sequence and translated concurrently. In some embodiments, the tag is cleavable and can be removed once the peptide is expressed and purified. The peptides may be linked directly, linked indirectly, or conjugated to the tag or agent. As used herein, the term “conjugate” refers to the joining of two entities by covalent bonds. The entities may be covalently bonded directly or through linking groups using standard synthetic coupling procedures. For example, two polypeptides may be linked together by simultaneous polypeptide expression, forming a fusion or chimeric protein. One or more amino acids may be inserted into the polypeptide to serve as a linking group (i.e., via incorporation of corresponding nucleic acid sequences into the vector). Other contemplated linking groups include polyethylene glycols or hydrocarbons terminally substituted with amino or carboxylic acid groups to allow for amide coupling with polypeptides having amino acids side chains with carboxylic acid or amino groups, respectively. Alternatively, the amino and carboxylic acid groups can be substituted with other binding partners, such as an azide and an alkyne group, which undergo copper catalyzed formation of triazoles.

In some embodiments, the peptides comprise multiple tags. For example, a second tag may be included to allow for easy capture of the peptides. Further tags contemplated include, for example, biotin (e.g., via a cysteine or lysine residue), a lipid molecule (e.g., via a cysteine residue), or a carrier protein or peptide. Attachment to tags, such as biotin, can be useful for associating the peptide with ligand receptors, such as avidin, streptavidin, polymeric streptavidin (see, e.g., US 2010/0081125 and US 2010/0267166, both of which are herein incorporated by reference), or neutravidin. Avidin, streptavidin, polymeric streptavidin, or neutravidin, in turn, can be linked to a detection/signaling moiety (e.g., an enzyme, such as horse radish peroxidase (HRP) or alkaline phosphatase (ALP) or β-galactosidase (β-GAL) or other moiety that can be visualized, such as a metallic nanomaterial such as nanoparticle, nanoplate, or nanoshell (e.g., colloidal gold), a fluorescent moiety, or a quantum dot) or a solid substrate (e.g., an Immobilon™ or nitrocellulose membrane or Porex® membrane). Alternatively, the peptides of the invention can be fused or linked to a ligand receptor, such as avidin, streptavidin, polymeric streptavidin, or neutravidin, thereby facilitating the association of the peptides with the corresponding ligand, such as biotin and any moiety (e.g., signaling moiety) or solid substrate attached thereto. Examples of other ligand-receptor pairs are well-known in the art and can similarly be used.

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc. Specifically, the proteins described herein are produced in tissue culture cells in vitro and are purified from the other cellular proteins to provide a purified protein that can be used as the capture agent in the immunoassay described herein.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Methods

In an aspect, the present invention provides methods of detecting cytomegalovirus infection in a subject. In some embodiments, the methods comprise: (a) providing an immunoassay comprising one or more capture reagents specific for CMV serotype A, B or C, preferably wherein the capture reagent is bound to a solid or semi-solid support; (b) contacting the immunoassay with a biological sample under conditions which allow for CMV antibodies if present in the biological sample to bind to the one or more capture reagents; (c) adding the detection reagent, and (d) detecting the complex formed between the capture reagent, CMV antibody and detection reagent within the immunoassay.

As used herein, “detecting” is defined as identifying the presence of a particular molecules within a sample. In some embodiments, detecting is performed by human observation. In some embodiments, detecting is performed by an automated device according to an established algorithm and involves no direct application of human observation or thought.

As used herein, “biological sample” or “test sample” refers to any sample of tissue, fluid, or material derived from a living organism. In some embodiments, the living organism is a primate. In some embodiments, the living organism is a human being, or Homo sapiens. Exemplary biological samples include, but are not limited to: saliva, blood, plasma, urine, feces, biopsies of organ tissues, e.g., spleen, liver, heart, lungs, etc. and any other biological samples known in the art.

As used herein, “complex” refers to two or more associated molecules. In some embodiments, a complex refers to capture reagent bound to anti-CMV antibodies or “CMV antibodies”. In some embodiments, a complex refers to capture reagents bound to CMV antibodies and further bound to a detection reagent.

In an aspect, the present invention provides methods for detecting the presence of CMV antibodies in a test sample. In some embodiments, the methods comprise the steps of: a) providing a test sample suspected of containing CMV antibodies; b) adding a quantity of the polypeptide selected from SEQ ID NO:4-6 to the sample, the quantity being sufficient to produce a detectable level of binding activity by CMV antibodies in the test sample; and c) detecting the presence of CMV bound to said polypeptide in the test sample by a detection reagent.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

In another aspect of the invention, there is provided a method for making an ELISA plate comprising the following steps: a) suspending the polypeptide of UL144 in buffer to a concentration suitable to produce a coating solution; b) adding the coating solution to each well; c) incubating the plate for at least about 12 hours at about 4° C.; d) removing the coating solution; e) blocking the plate; f) washing the plate; thereby preparing the ELISA assay plate. In some embodiments, the UL144 polypeptide is encoded by a nucleotide sequence selected from SEQ ID NO: 7-9.

In another embodiment, the disclosure provides a method for detecting the presence of CMV serotypes in a biological sample comprising the following steps: a) providing the ELISA plate described herein; b) providing at least an experimental sample, at least one negative control sample, and at least one positive control sample containing antibodies specific to at least one CMV serotype; c) diluting the sample(s) in blocking buffer; d) adding an amount of diluted sample(s) into the wells of the plate; e) incubating the plates; f) washing the wells of the plates to remove the diluted sample(s); g) developing the plates with a detection agent; and i) reading the plate, wherein determining that CMV antibody is present where the sample has significantly more signal than the negative control samples, thereby detecting the presence of CMV in the sample(s).

In yet another embodiment, the disclosure provides a method for detecting the presence of CMV antibodies in a test sample, comprising the steps of: a) providing a test sample suspected of containing CMV antibodies; b) adding a quantity of the polypeptide to the test sample, the quantity being sufficient to produce a detectable level of binding activity by anti-UL144 antibodies in the test sample; and c) detecting the presence of UL144 antibodies bound to said polypeptide in the test sample.

In another embodiment, the disclosure provides a method for detecting the presence of CMV UL144 antibodies in a test sample, comprising the steps of: a) providing a test sample suspected of containing CMV antibodies; b) adding a quantity of an immunogenic fragment of the polypeptide to the test sample, the quantity being sufficient to produce a detectable level of binding activity by anti-UL144 antibodies in the test sample; and c) detecting the presence of UL144 antibodies bound to said polypeptide in the test sample.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1: A Novel Multiplexed Enzyme-Linked Immunosorbent Assay for the Detection of IgG Seroreactivity to Cytomegalovirus (CMV) UL144

In the following example, the inventor describes exemplary methods and assays developed to differentiate between antibodies developed in response to vaccination against CMV and antibodies developed in response to natural infection with CMV.

Immunization with CMV vaccines based on the AD169 strain may elicit neutralizing antibody responses that are indistinguishable from neutralizing antibodies to natural infection. To address this limitation, the inventor aimed to develop a new multiplex ELISA assay for CMV protein UL144. The UL144 gene, present in the UL/b′ boundary of the CMV genome, is present in clinical isolates but absent in AD169 and other laboratory-adapted strains (7). The inventor and others defined three major, distinct UL144 genotypes from multiple original samples and clinical isolates (8-11). The three major UL144 genotypes (A, B, and C) and recombinants have similar distribution in different geographical locations (9, 12, 13), and specific genotypes may be associated with severe sequelae of congenital CMV infection (8, 12). UL144 is a membrane-anchored glycoprotein that is largely intracellular, but it is also found on the cell surface when overexpressed (14, 15). This report characterizes an ELISA method for measurement of the serum antibody response to the three UL144 types.

Materials and Methods

UL144 Protein Expression and Purification

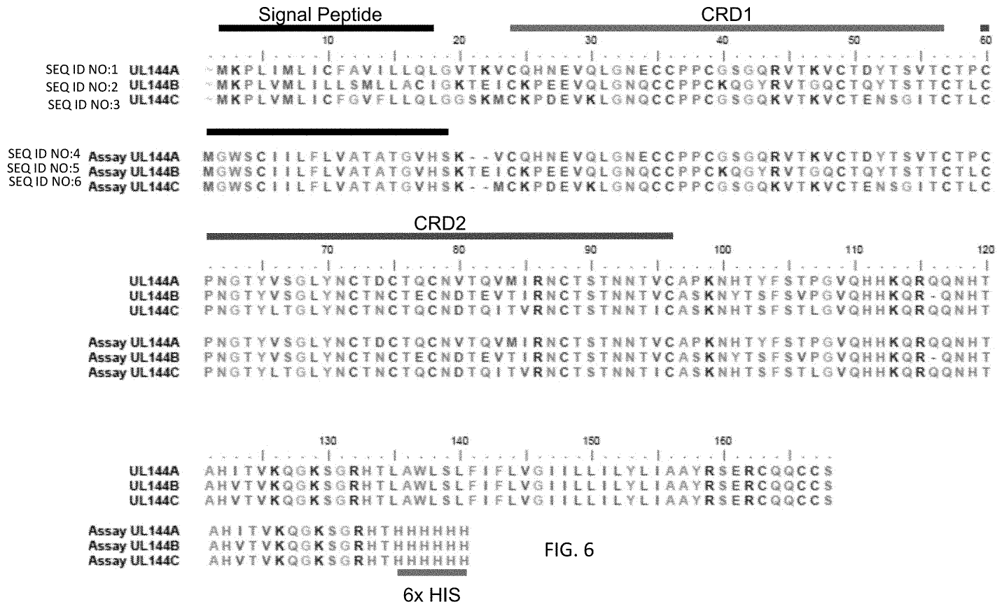

UL144 A, B, and C genotypes were codon optimized for expression and secretion by mammalian cells, and a C-terminus 6×Histidine tag was added (Genscript Biotech Corp, Piscataway, N.J., Gene sequences were subcloned into pcDNA3.4. The resulting plasmids were transfected into Expi293F cells grown in serum-free Expi293™ expression medium (Thermo Fisher Scientific, Waltham, Mass.) and maintained at 37° C. with 8% CO2 on an orbital shaker. DNA and ExpiFectamine™ 293 reagent (Thermo) were mixed and added into the cells. Cell culture supernatants were collected, centrifuged, and purified on an Ni-NTA column. Sample purity was evaluated using a 4-20% gradient SDS-PAGE gel, and protein concentration was determined by Bradford assay. Protein sequences are provided in FIG. 6.

Serum Samples and Ethics Statement

CMV seropositive and seronegative sera were collected from the Children's Wisconsin Clinical Microbiology Laboratory and the Wisconsin Diagnostic Laboratories (WDL, March 2019-March 2021). The Institutional Review Board determined that this is not Human Research, since all samples were de-identified, and there was no way to link a coded sample to a specific patient. CMV IgM and IgG were determined by an ELISA assay (Zeus Scientific, Branchburg, N.J.), which measures serum reactivity to antigens derived from the cell-culture-passaged CMV strain AD169. IgG positive, or both IgG and IgM negative samples were analyzed by the anti-UL144 ELISA assay. To test for antigen cross-reactivity, HSV1 seropositive, CMV seronegative samples were obtained from Children's Wisconsin. Briefly, serum samples were allowed to clot for 1 hour, followed by centrifugation for 10 minutes at 3,000×g. Samples were stored at 4° C. overnight, then frozen at −20° C. For the anti-UL144 ELISA assay, serum samples were thawed, gently resuspended, aliquoted, and refrozen to minimize freeze-thaw cycles. Samples used for validation of the assay were assayed immediately after thawing. Once a serum aliquot was thawed, it was kept at 4° C. until depleted.

To confirm the specificity of antigen recognition by the ELISA assay, deidentified CMV PCR positive serum samples were selected for UL144 sequencing. Viral load was measured at WDL using the Cobas® CMV PCR assay (Roche Diagnostic Corporation, Indianapolis, Ind.). DNA was isolated from serum, and UL144 PCR and sequencing were performed using primers previously reported (9). The resulting DNA sequences were translated to amino acid sequences and compared to reference UL144 A, B and C using Clustal Omega analysis.

UL144 IgG ELISA

All volumes were 50 μl per well unless otherwise indicated. Immulon IIHB 96-well microplates (3455, Thermo Fisher Scientific) were coated with a multiplexed antigen mixture of 0.3125 μg/mL UL144 A, 0.625 μg/mL UL144 B and 0.078125 μg/mL of UL144 C protein diluted in phosphate buffered saline (PBS) pH 7.4 (114-058-101, Quality Biologicals, Gaithersburg, Md.), sealed and incubated overnight at 4° C. For serum-specific background noise (SSBN) reduction, each plate also included one well per serum sample plated in PBS (no antigen). Following overnight incubation, the plates were washed 5 times with 300 μl of buffer [0.05% PBS (119-069-151, Quality Biological)/Tween-20 (P9416, Sigma) (PBS-T)] between each incubation step using an ELx50 microplate washer (BioTek, Winooski, Vt.). To examine the stability of antigen-coated plates post-freezing, PBS-T was removed, and the plates were patted dry, sealed, and stored upright at −80° C. for later evaluation. The following incubation steps were performed at 37° C. with shaking at 250 RPM: blocking, serum dilution, secondary antibody, and streptavidin-horseradish peroxidase (SA-HRP). Plates were blocked in 150 μl blocking buffer (5% BSA (A2394, Sigma) and 5% blocking-grade buffer (1706404, Bio-Rad) in PBS for two hours, followed by washing 5 times with PBS-T (16). Serum samples were diluted 1:40 in blocking buffer and incubated on the plate for one hour. Following washing, plates were incubated for one hour with biotin-conjugated anti-Human IgG antibody (109-065-088, Jackson Immunoresearch, West Grove, Pa.) diluted 1:40,000 in blocking buffer. After washing, the plates were probed with SA-HRP (P121130, Thermo Fisher Scientific) diluted 1:16,000 in blocking buffer for 30 minutes. Plates were developed with 100 μl of 3,3′,5,5′ Tetramethylbensizidine (421101, TMB) 2-component substrate (Biolegend, San Diego, Calif.) for ten minutes at room temperature and the reaction was stopped using 100 μl of 2M H₂SO₄ (BDH3068, VWR, Radnor, Pa.). Absorbance was immediately measured at 450 nm on a Spectra-Max plate reader (Molecular Devices, San Jose, Calif.).

Statistical Analysis

Each plate included 1-2 positive and 2-3 negative control samples as determined by the clinical CMV ELISA assay. Three antigen-coated wells were used for each serum sample, unless otherwise indicated. SSBN reduction was determined by subtracting the optical density (OD) value of the PBS-coated well from the value of the antigen-coated well for each serum. For initial studies, the cutoff value for each plate was determined using the average values of the negative samples normalized using SSBN plus three times the standard deviation of the negative samples. Summary statistics were reported as mean (standard deviation, SD), median (interquartile range), or number (percent) where relevant. Predictive values of the UL144 ELISA used the clinical CMV ELISA as a “gold standard”, defined by the Federal Drug Administration: www.federalregister.gov/documents/2007/03/13/E7-4453/guidance-for-industry-and-food-and-drug-administration-staff-statistical-guidance-on-reporting. Although no “gold standard” for CMV seroreactivity exists, sensitivity, specificity, positive predictive value (PPV) and negative predictive value (NPV) are provided. Receiver operating characteristic curves (ROC) and area under the curve (AUC) were generated to obtain thresholds. An estimated prevalence of CMV seropositivity of 40% was used for calculating the NPV and PPV, based on population data and the collection samples (children and adults) (17). Predictive values are given with exact+95% Clopper-Pearson intervals.

Results

Optimization of UL144 ELISA

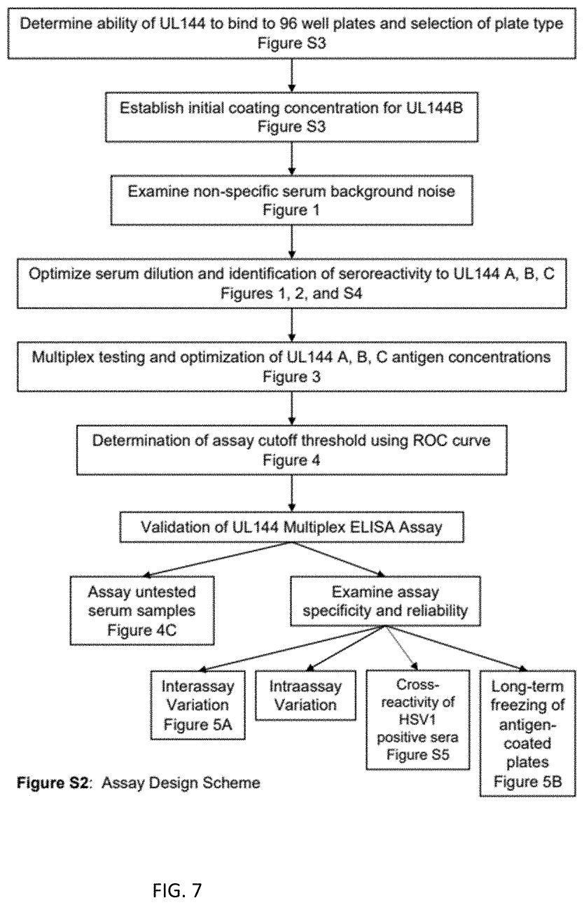

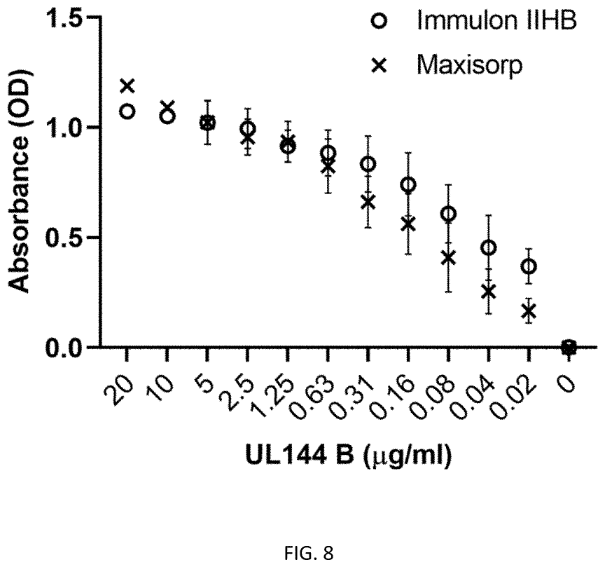

The overall assay design scheme is outlined in FIG. 8. UL144 B was used in the initial experiments to determine the optimal conditions for the assay. The conditions for UL144 A and C were subsequently evaluated in the same manner. A checkerboard pattern was used in optimization steps unless otherwise noted.

Selection of ELISA Plate

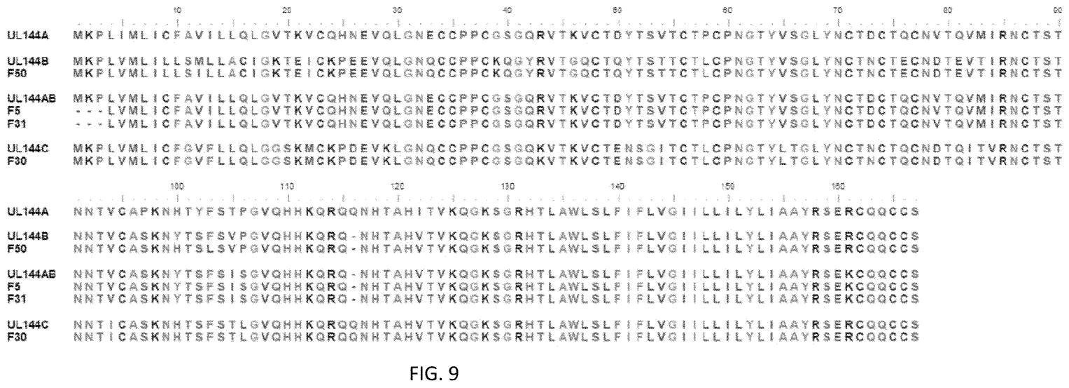

The N-terminal 6×HIS tag was used to detect binding of UL144 B to the Immulon IIHB and Maxisorp 96-well plates. At the lower coating antigen concentrations UL144 had greater affinity for Immulon IIHB plates than Maxisorp plates, as shown by higher OD values (FIG. 9), therefore, Immulon IIHB plates were selected.

Determination of Coating Antigen Concentration

Plateau of the OD values was used to determine the saturation of the coating antigen on the plate. The plateau occurred between 0.325 μg/ml to 1.25 μg/ml, and 0.625 μg/ml was used for further optimization (FIG. 8). UL144 A and C coating antigen concentrations were determined in a similar manner. Initial studies used 1.25 μg/ml UL144 A and 0.3125 μg/ml UL144 C.

The specificity UL144 B antigen recognition was confirmed using a PCR-positive serum sample which was seroreactive to UL144 B only (F50). The amino acid sequence of UL144 from serum was 99% identical to the reference UL144 B sequence (FIG. 9).

Background Noise Reduction

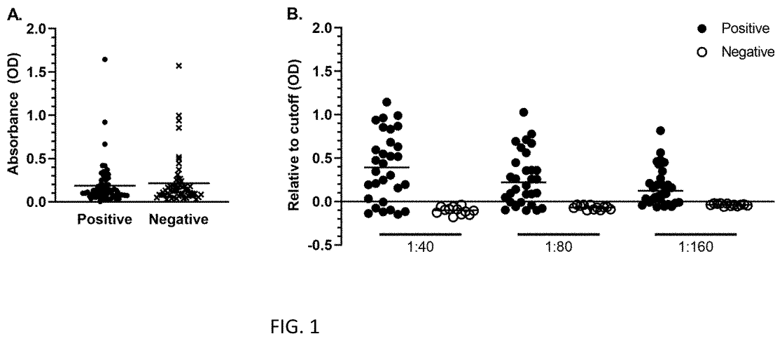

Recent reports have demonstrated that non-specific binding of serum can negatively impact the sensitivity and specificity of ELISA tests (18, 19). 56 CMV-positive and 62 CMV-negative serum samples defined by the clinical ELISA assay were diluted 1:40 in blocking buffer to test the capacity of serum to bind to the plate in the absence of coating antigen (FIG. 1A). OD values measured following incubation with serum only and no protein coating ranged up to 1.7 OD compared to wells with no serum. Therefore, subsequent assay optimization was performed using SSBN reduction, where the OD value of an uncoated well is subtracted from the OD value of wells coated with UL144.

Serum Dilution and Identification of Seroreactivity to UL144 A, B, C

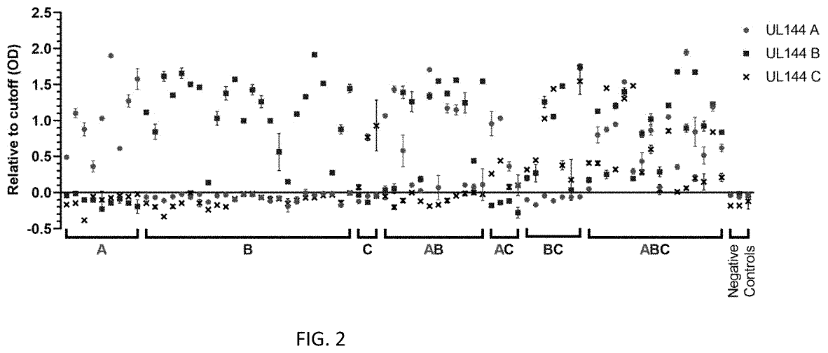

An initial screen of 42 (30 CMV-seropositive and 12 CMV-seronegative) samples was used to test for reactivity to UL144 B (FIG. 1). Three serum dilutions were compared: 1:40, 1:80 and 1:160. Six of the samples below the cutoff for the 1:40 dilution were subsequently determined to be reactive to another UL144 serotype. Excluding non-type B samples, the best sensitivity (96.8%) and specificity (100%) was observed at 1:40 serum dilution. Seventy-five CMV-positive clinical ELISA samples were evaluated for reactivity to UL144 A, B, or C (FIG. 2). All samples were seroreactive to at least one antigen. Nine samples were UL144 A positive (12%), 24 were UL144 B positive (32%), and three were UL144 C positive (4%). Thirty-nine samples showed reactivity to more than one UL144 antigen, likely due to antibody recognition of shared epitopes, infection with a recombinant UL144 strain, or infection with more than one UL144 subtype. Twelve (16%) were reactive to UL144 A and B, seven (9.3%) to UL144 B and C, four to UL144 A and C (5.3%), and 16 (21.3%) to A, B and C. Two samples reactive to UL144 A and B were sequenced (F5 and F31), and identified as genotype A/B. One serum sample, reactive to UL144 A, B, and C (F30) was identified as UL144 C by Sanger sequencing (FIG. 9).

Serum samples with the highest OD values were used for further refinement of UL144 A and UL144 C conditions in a similar manner to UL144 B.

Multiplex Testing and Optimization of UL144 A, B, C Coating Antigens

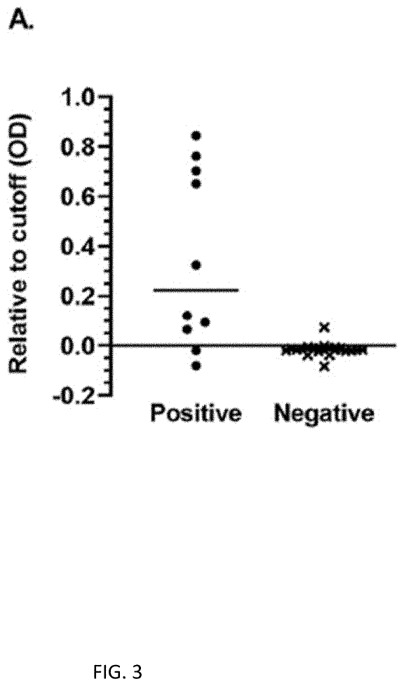

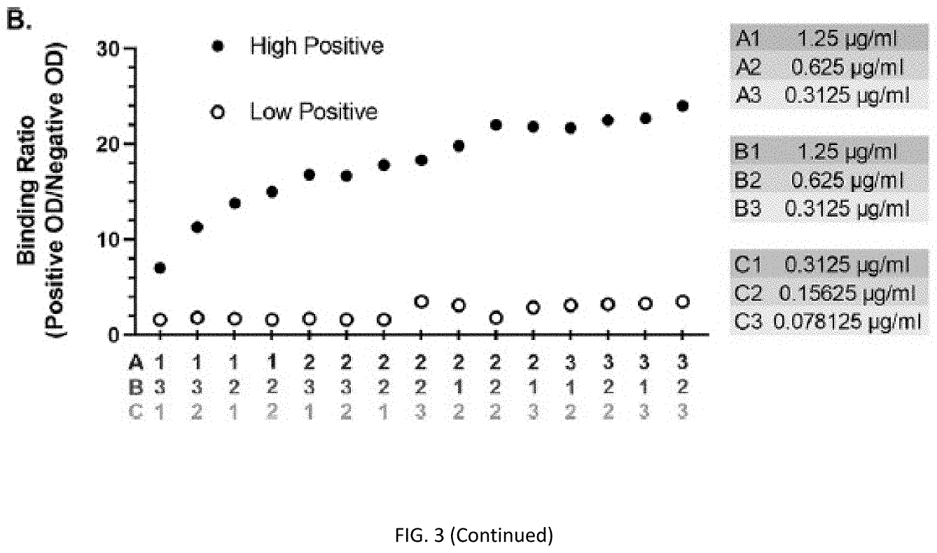

Using the previously determined coating antigen concentrations for UL144 A, B, and C, and 1:40 serum dilution, a multiplex ELISA assay was performed on select CMV serum samples (FIG. 3A). Three CMV negative serum samples were used to determine the cutoff. The multiplex assay was found to have a sensitivity of 80%, and a specificity of 95.8%. Combinations of different coating antigen concentrations of UL144 A, B, and C were evaluated against a set of three serum samples. Two CMV positive samples, including one which previously tested positive but was near the cutoff (low positive), and one CMV negative serum sample were used to evaluate different coating antigen combinations (FIG. 3B, left). The best conditions were found to be with lower concentrations of UL144 A and C, and higher concentrations of UL144 B. The highest binding ratio for both the low and high positive samples was observed with 0.3125 μg/ml UL144 A, 0.625 ug/ml UL144 B, and 0.078125 μg/ml UL144 C (FIG. 3B).

Determination of Assay Threshold Using ROC Curve

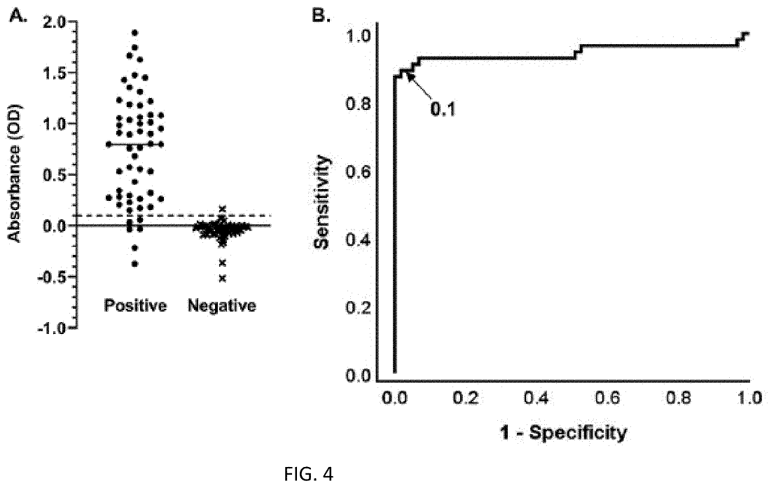

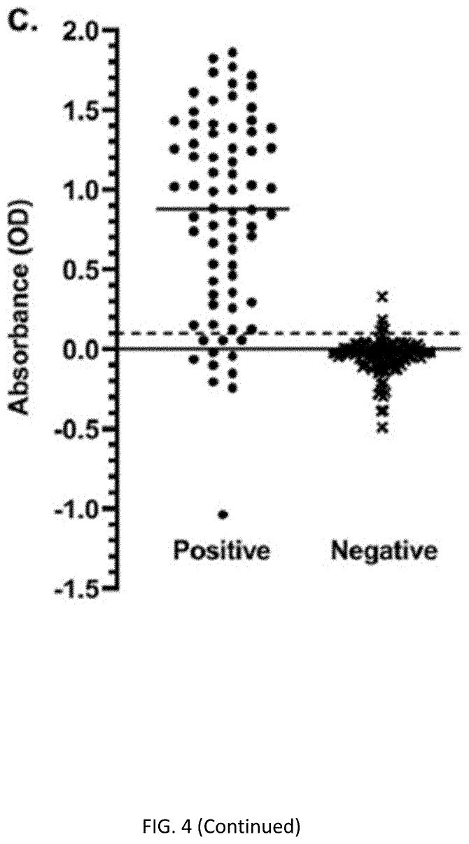

114 sera were used to generate an ROC curve, 55 were clinical ELISA seropositive and 59 were clinical ELISA seronegative (FIG. 4A). The assay sensitivity was 89.1% (95% CI 77.8%-95.9%), the specificity was 98.3% (90.9%-100%), PPV was 97.2% (83.4%-99.6%) and the NPV was 93.1% (86.4%-96.6%). The area under the ROC curve (AUC) was 0.945. A threshold of 0.1 was determined (FIG. 4B).

Validation of Multiplex Assay

A set of 189 previously untested serum samples (72 clinical ELISA positive and 116 clinical ELISA negative) were tested for CMV UL144-specific antibodies using the multiplexed ELISA assay (FIG. 4C). The sensitivity, specificity, PPV and NPV were: 84.93% (74.64% to 92.23%), 95.69% (90.23% to 98.59%), 92.93% (84.72% to 96.89%), and 90.50% (84.66% to 94.27%). Based on the total number of samples (303), the overall sensitivity, specificity, PPV and NPV were 86.72% (95% CI 79.59% to 92.07%), 96.57% (92.69% to 98.73%), 94.40% (88.45% to 97.38%), and 91.60% (87.50% to 94.44%).

Assay Specificity and Reliability

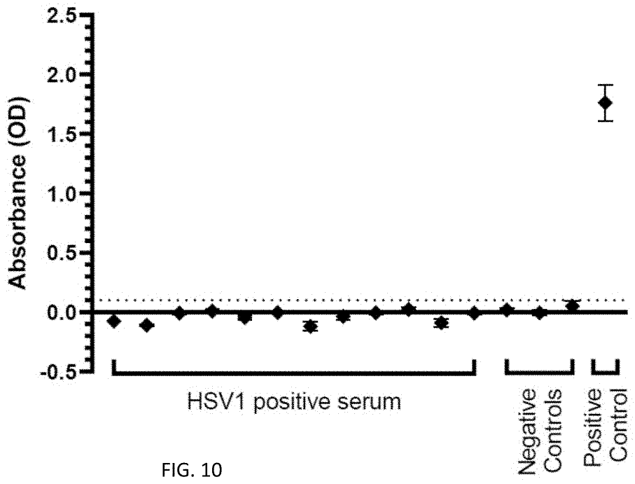

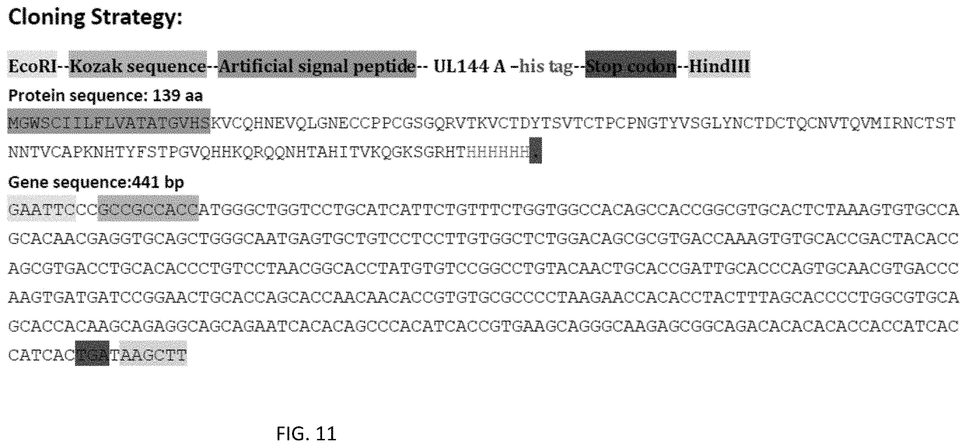

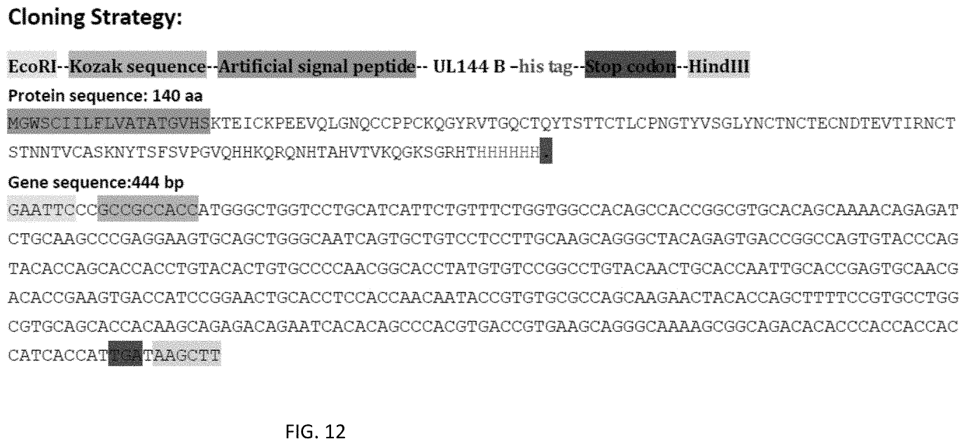

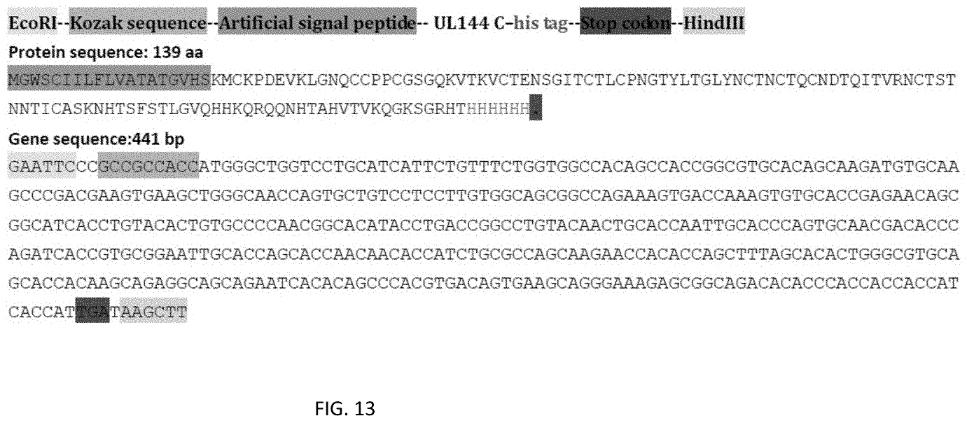

To address potential cross-reactivity with other pathogens that express TNF receptor homologs and to further confirm the specificity of the UL144 ELISA assay, the inventor tested sera that was herpes simplex 1 (HSV1) positive and CMV negative. The herpes virus entry mediator (HVEM) is a TNF receptor homolog related to UL144 (20). Twelve HSV1 seropositive, CMV seronegative samples were tested for UL144 reactivity, and none were found to be reactive (FIG. 10).

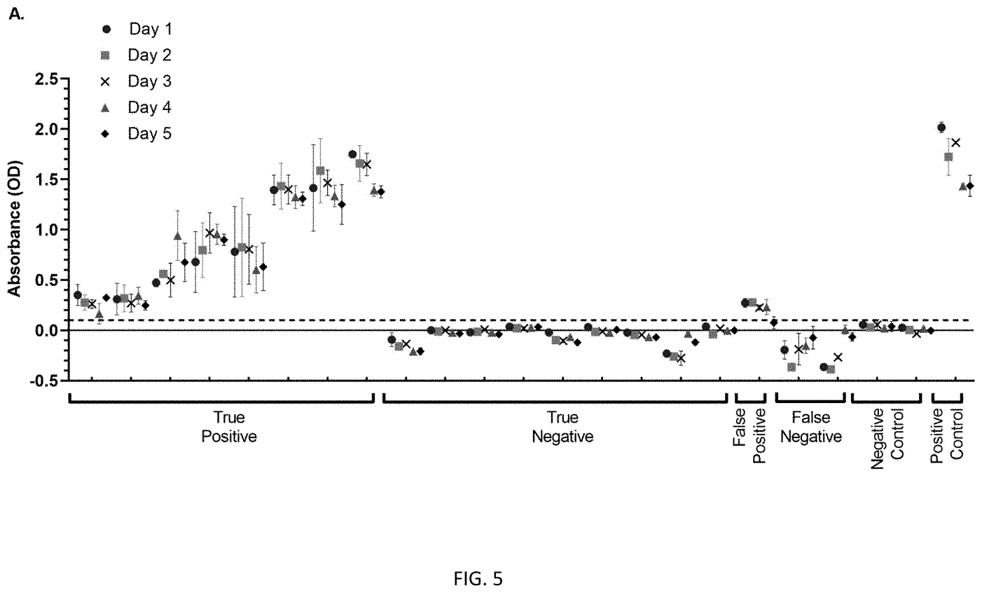

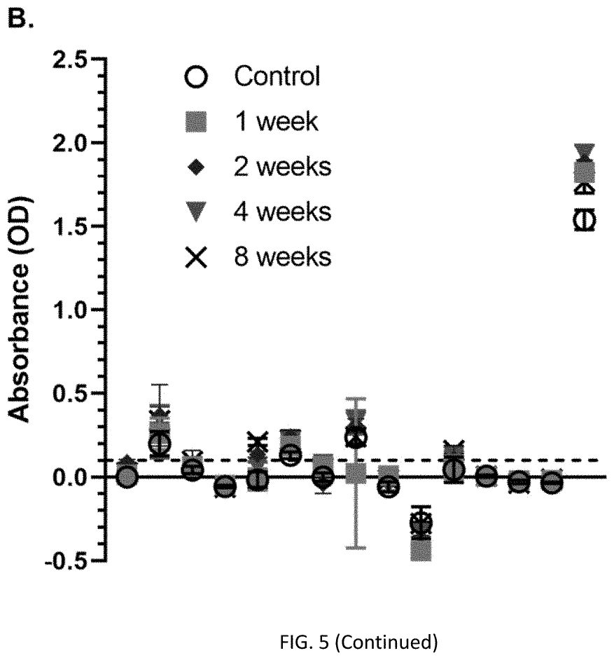

The inter-assay reliability was determined by testing 20 serum samples of unknown serostatus with the multiplex assay on five separate occasions (FIG. 5A). The inter-assay median coefficient of variation was 0.06 (IQR −0.56, 0.2). The intraassay reliability was determined by plating five replicate wells of seven serum samples on one plate. The median coefficient of variation for all samples was 0.171 (IQR 0.038, 0.302).

Long-Term Freezing of Plates

To determine the long-term storage potential of antigen-coated plates, 15 samples were tested after coating the plates overnight and long-term storage at −80° C. as described. One plate was prepared without freezing and served as a control. Plates were frozen for 1, 2, 4 or 8 weeks. After thawing, the assay was repeated under identical conditions to the control plate. Freezing the plates had no effects on the results, and high agreement was observed at all time points (FIG. 5B).

Discussion

Our new multiplex UL144 ELISA assay showed high correlation with the clinical CMV ELISA with an overall sensitivity of 86.72%, specificity of 96.57%, PPV of 94.40% and NPV of 91.60%. All three major UL144 antigens elicited antibody responses. Several ELISA assays have been developed for detection of IgG to CMV glycoproteins and provide specific assays in the setting of CMV vaccine trials (4). While our assay is not intended to replace available CMV ELISA assays, it has several advantages. In contrast to the commercial assays, it is based on UL144, a protein that is deleted from laboratory adapted strains, but present in clinical isolates. Therefore, epidemiological studies, specifically vaccine trials based on laboratory adapted strains, may take advantage of this assay to discriminate response to natural infection from that of vaccine. It can also provide serotype specific information for UL144 A, B and C, and can complement and confirm molecular epidemiology studies. Although UL144 is expressed intracellularly, antibody responses to UL144 were suggested (15). UL144 provides a novel candidate for antibody responses. It is amongst the open reading frames in the UL/b′ boundary of CMV clinical isolates and encodes a type I transmembrane protein that contains a leader peptide, two cysteine rich domains, membrane extension region, transmembrane domain, and a short cytoplasmic tail. It is heavily glycosylated and expressed early after infection (15). In all clinical samples, identity is noted in the number and positioning of cysteines, as well as the transmembrane and cytoplasmic domains. Studies of the immunological functions of UL144 during CMV infection reveal complex activities. UL144 has sequence similarity to the TNFα receptor (TNFαR) superfamily (21). It has 37% sequence homology to Herpes Virus Entry Mediator (HVEM), however, it lacks a third cysteine rich domain, which has been shown to contain important ligand receptor contacts. Despite its sequence homology to HVEM, the inventor did not detect cross reactivity of antibody response amongst 12 HSV-positive CMV-negative sera. However, the inventor cannot rule out cross reactivity to other viruses encoding TNF receptors, such as Poxviruses (vaccinia, variola, cowpox and monkeypox), though their diagnosis should be supported by clinical findings (22). UL144 has not shown to bind any of the TNF-related ligands, and it lacks a death domain (23). Yet, it was reported a potent activator of NF-κB-induced transcription in a TRAF-6 dependent manner. NF-κB activation was concomitant with increased expression of CCL22, a chemokine that attracts Th2 cells, which in turn induce humoral immune response (24). An interaction between B and T lymphocyte attenuator (BTLA) and HVEM suggested HVEM may act as a negative regulator by binding to BTLA (25). The ectodomain of UL144 also interacts with BTLA and inhibits T cell proliferation in vitro, suggesting UL144 mimics the inhibitory co-signaling function of HVEM and may modulate T cell responses (20). In a cobort of kidney transplant patients (CMV donor positive recipient negative) who acquired primary CMV infection, all developed robust interferon γ (IFN γ) responses to a pool of CMV peptides that included UL44. However, supernatants collected from UL144-stimulated T cells did not exhibit antiviral activity, suggesting factors other than IFNγ may be important to the secreted anti-CMV immune response (26). The overall effects of ULL144 on T cell functions may result in IgG responses to UL144, as detected by our assay.

Genetic analysis of UL144 from human samples has shown three major genotypes: A, B and C, and several recombinants, based on the UL144 signal peptide and the first cysteine rich domain, resulting in distinct protein sequences that differ by 20-27% (12). The sequence variation in UL144 may contribute to some of the negative antibody responses previously described (15). Notably, the ELISA assay detected reactivity to one or more UL144 type in samples from Wisconsin, which may indicate serological response to UL144 recombinants, infection with several UL144 strains that are not detected by population-based Sanger sequencing, or antibody recognition of shared epitopes. The benefit of using UL144 as an antigen for the ELISA assay is its presence in all clinical isolates (as in natural infection) and absence from laboratory adapted CMV strains used for commercial CMV ELISA assays.

A recent CMV vaccine based on the live attenuated AD169 strain in which the pentameric complex gH/gL/pUL128-131 has been restored was found immunogenic in animal models and phase I vaccine trials (27). In a phase I clinical trial, V160 was administered at 0, 1, and 6 months and induced neutralizing antibody titers equal to or higher than those observed in naturally occurring, unvaccinated seropositive subjects with no virus shedding.

The multiplex UL144 ELISA assay may provide an important tool for detection of natural CMV infection during CMV vaccine trials, such as the one described above. The assay can detect antibody reactivity to all UL144 antigens, with high specificity, positive and negative predictive values. The assay may also provide an important tool for epidemiological studies of CMV strains. Future prospective studies will determine the duration of antibody responses to UL144.

### REFERENCES

1. Razonable R R, Inoue N, Pinninti S G, Boppana S B, Lazzarotto T, Gabrielli L, Simonazzi G, Pellett P E, Schmid D S. 2020. Clinical Diagnostic Testing for Human Cytomegalovirus Infections. J Infect Dis 221:S74-S85.
2. Guerra B, Lazzarotto T, Quarta S, Lanari M, Bovicelli L, Nicolosi A, Landini M P. 2000. Prenatal diagnosis of symptomatic congenital cytomegalovirus infection. Am J Obstet Gynecol 183:476-482.
3. Forman M S, Vaidya D, Bolorunduro O, Diener-West M, Pass R F, Arav-Boger R. 2017. Cytomegalovirus Kinetics Following Primary Infection in Healthy Women. J Infect Dis 215:1523-1526.
4. Novak Z, Ross S A, Patro R K, Pati S K, Reddy M K, Purser M, Britt W J, Boppana S B. 2009. Enzyme-linked immunosorbent assay method for detection of cytomegalovirus strain-specific antibody responses. Clin Vaccine Immunol 16:288-290.
5. Hackett D J, Zhang C, Stefanescu C, Pass R F. 2010. Enzyme-linked immunosorbent assay for measurement of cytomegalovirus glycoprotein B antibody in serum. Clin Vaccine Immunol 17:836-839.
6. Pagnon A, Piras F, Gimenez-Fourage S, Dubayle J, Arnaud-Barbe N, Hessler C, Caillet C. 2017. Optimized enzyme-linked immunosorbent assay for detecting cytomegalovirus infections during clinical trials of recombinant vaccines. J Clin Virol 96:99-104.
7. Cha T A, Tom E, Kemble G W, Duke G M, Mocarski E S, Spaete R R. 1996. Human cytomegalovirus clinical isolates carry at least 19 genes not found in laboratory strains. J Virol 70:78-83.
8. Waters A, Hassan J, De G C, Kissoon G, Knowles S, Molloy E, Connell J, Hall W W. 2010. Human cytomegalovirus UL144 is associated with viremia and infant development sequelae in congenital infection. J Clin Microbiol 48:3956-3962.
9. Arav-Boger R, Battaglia C A, Lazzarotto T, Gabrielli L, Zong J C, Hayward G S, Diener-West M, Landini M P. 2006. Cytomegalovirus (CMV)-encoded UL144 (truncated tumor necrosis factor receptor) and outcome of congenital CMV infection. J Infect Dis 194:464-473.
10. Lurain N S, Kapell K S, Huang D D, Short J A, Paintsil J, Winkfield E, Benedict C A, Ware C F, Bremer J W. 1999. Human cytomegalovirus UL144 open reading frame: sequence hypervariability in low-passage clinical isolates. J Virol 73:10040-10050.
11. Picone O, Costa J M, Chaix M L, Ville Y, Rouzioux C, Leruez-Ville M. 2005. Human cytomegalovirus UL144 gene polymorphisms in congenital infections. J Clin Microbiol 43:25-29.
12. Arav-Boger R, Willoughby R E, Pass R F, Zong J C, Jang W J, Alcendor D, Hayward G S. 2002. Polymorphisms of the cytomegalovirus (CMV)-encoded tumor necrosis factor-alpha and beta-chemokine receptors in congenital CMV disease. J Infect Dis 186:1057-1064.
13. Murthy S, Hayward G S, Wheelan S, Forman M S, Ahn J H, Pass R F, Arav-Boger R. 2011. Detection of a single identical cytomegalovirus (CMV) strain in recently seroconverted young women. PLoS One 6:e15949.
14. Poole E, Walther A, Raven K, Benedict C A, Mason G M, Sinclair J. 2013. The myeloid transcription factor GATA-2 regulates the viral UL144 gene during human cytomegalovirus latency in an isolate-specific manner. J Virol 87:4261-71.
15. Benedict C A, Butrovich K D, Lurain N S, Corbeil J, Rooney I, Schneider P, Tschopp J, Ware C F. 1999. Cutting edge: a novel viral TNF receptor superfamily member in virulent strains of human cytomegalovirus. J Immunol 162:6967-6970.
16. Xiao Y, Isaacs S N. 2012. Enzyme-linked immunosorbent assay (ELISA) and blocking with bovine serum albumin (BSA)—not all BSAs are alike. J Immunol Methods 384:148-51.
17. Staras S A, Dollard S C, Radford K W, Flanders W D, Pass R F, Cannon M I. 2006. Seroprevalence of cytomegalovirus infection in the United States, 1988-1994. Clin Infect Dis 43:1143-1151.
18. Terato K, Do C, Chang J, Waritani T. 2016. Preventing further misuse of the ELISA technique and misinterpretation of serological antibody assay data. Vaccine 34:4643-4644.
19. Waritani T, Chang J, McKinney B, Terato K. 2017. An ELISA protocol to improve the accuracy and reliability of serological antibody assays. MethodsX 4:153-165.
20. Cheung T C, Humphreys I R, Potter K G, Norris P S, Shumway H M, Tran B R, Patterson G, Jean-Jacques R, Yoon M, Spear P G, Murphy K M, Lurain N S, Benedict C A, Ware C F. 2005. Evolutionarily divergent herpesviruses modulate T cell activation by targeting the herpesvirus entry mediator cosignaling pathway. Proc Natl Acad Sci USA.
21. Marsters S A, Ayres T M, Skubatch M, Gray C L, Rothe M, Ashkenazi A. 1997. Herpesvirus entry mediator, a member of the tumor necrosis factor receptor (TNFR) family, interacts with members of the TNFR-associated factor family and activates the transcription factors NF-kappaB and AP-1. J Biol Chem 272:14029-14032.
22. Alcami A. 2003. Viral mimicry of cytokines, chemokines and their receptors. Nat Rev Immunol 3:36-50.
23. Poole E, King C A, Sinclair J H, Alcami A. 2006. The UL144 gene product of human cytomegalovirus activates NFkappaB via a TRAF6-dependent mechanism. EMBO J 25:4390-4399.
24. Poole E, Atkins E, Nakayama T, Yoshie O, Groves I, Alcami A, Sinclair J. 2008. NF-kappaB-mediated activation of the chemokine CCL22 by the product of the human cytomegalovirus gene UL144 escapes regulation by viral IE86. J Virol 82:4250-4256.
25. Gonzalez L C, Loyet K M, Calemine-Fenaux J, Chauhan V, Wranik B, Ouyang W, Eaton D L. 2005. A coreceptor interaction between the CD28 and TNF receptor family members B and T lymphocyte attenuator and herpesvirus entry mediator. Proc Natl Acad Sci USA 102:1116-1121.
26. Houldcroft C J, Jackson S E, Lim E Y, Sedikides G X, Davies E L, Atkinson C, McIntosh M, Remmerswaal E B M, Okecha G, Bemelman F J, Stanton R J, Reeves M, Wills M R. 2020. Assessing Anti-HCMV Cell Mediated Immune Responses in Transplant Recipients and Healthy Controls Using a Novel Functional Assay. Front Cell Infect Microbiol 10:275.
27. Wang D, Freed D C, He X, Li F, Tang A, Cox K S, Dubey S A, Cole S, Medi M B, Liu Y, Xu J, Zhang Z Q, Finnefrock A C, Song L, Espeseth A S, Shiver J W, Casimiro D R, Fu T M. 2016. A replication-defective human cytomegalovirus vaccine for prevention of congenital infection. Sci Transl Med 8:362ra145.

TABLE 1

| Name | Sequence |
|---|---|
| UL144A | MKPLIMLICFAVILLQLGVTKVCQHNEVQL |
| (SEQ ID | GNECCPPCGSGQRVTKVCTDYTSVTCTPCP |
| NO: 1) | NGTYVSGLYNCTDCTQCNVTQVMIRNCTST |
| | NNTVCAPKNHTYFSTPGVQHHKQRQQNHTA |
| | HITVKQGKSGRHTLAWLSLFIFLVGIILLI |
| | LYLIAAYFQCCS |
| UL144B | MKPLVMLILLSMLLACIGKTEICKPEEVQL |
| (SEQ ID | GNQCCPPCKQGYRVTGQCTQYTSTTCTLCP |
| NO: 2) | NGTYVSGLYNCTNCTECNDTEVTIRNCTST |
| | NNTVCASKNYTSFSVPGVQHHKQRQNHTAH |
| | VTVKQGKSGRHTLAWLSLFIFLVGIILLIL |
| | YLIAAYRSERCQQCCS |
| UL144C | MKPLVMLICFGVFLLQLGGSKMCKPDEVKL |
| (SEQ ID | GNQCCPPCGSGQRVTKVCTENSGITCTLCP |
| NO: 3) | NGTYLTGLYNCTNCTQCNDTQITVRNCTST |
| | NNTICASKNHTSFSTLGVQHHKQRQQNHTA |
| | HVTVKQGKSGRHTLAWLSLFIFLVGIILLI |
| | LYLIAAYRSERCQQCCS |
| Assay | MGWSCIILFLVATATGVHSK--VCQHNEVQ |
| UL144A | LGNECCPPCGSGQRVTKVCTDYTSVTCTPC |
| (SEQ ID | PNGTYVSGLYNCTDCTQCNVTQVMIRNCTS |
| NO: 4) | TNNTVCAPKNHTYFSTPGVQHHKQRQQNHT |
| | AHITVKQGKSGRHTHHHHHH |
| Assay | MGWSCIILFLVATATGVHSKTEICKPEEVQ |
| UL144B | LGNQCCPPCKQGYRVTGQCTQYTSTTCTLC |
| (SEQ ID | PNGTYVSGLYNCTNCTECNDTEVTIRNCTS |
| NO: 5) | TNNTVCASKNYTSFSVPGVQHHKQR-QNHT |
| | AHVTVKQGKSGRHTHHHHHH |
| Assay | MGWSCIILFLVATATGVHSK--MCKPDEVK |
| UL144C | LGNQCCPPCGSGQRVTKVCTENSGITCTLC |
| (SEQ ID | PNGTYLTGLYNCTNCTQCNDTQITVRNCTS |
| NO: 6) | TNNTICASKNHTSFSTLGVQHHKQRQQNHT |
| | AHVTVKQGKSGRHTHHHHHH |
| Codon | GAATTCCCGCCGCCACCATGGGCTGGTCCT |
| optimized | GCATCATTCTGTTTCTGGTGGCCACAGCCA |
| DNA | CCGGCGTGCACAGCAAAACAGAGATCTGCA |
| for | AGCCCGAGGAAGTGCAGCTGGGCAATCAGT |
| UL144A | GCTGTCCTCCTTGCAAGCAGGGCTACAGAG |
| (SEQ ID | TGACCGGCCAGTGTACCCAGTACACCAGCA |
| NO: 7) | CCACCTGTACACTGTGCCCCAACGGCACCT |
| | ATGTGTCCGGCCTGTACAACTGCACCAATT |
| | GCACCGAGTGCAACGACACCGAAGTGACCA |
| | TCCGGAACTGCACCTCCACCAACAATACCG |
| | TGTGCGCCAGCAAGAACTACACCAGCTTTT |
| | CCGTGCCTGGCGTGCAGCACCACAAGCAGA |
| | GACAGAATCACACAGCCCACGTGACCGTGA |
| | AGCAGGGCAAAAGCGGCAGACACACCCACC |
| | ACCACCATCACCATTGATAAGCTT |
| Codon | GAATTCCCGCCGCCACCATGGGCTGGTCCT |
| optimized | GCATCATTCTGTTTCTGGTGGCCACAGCCA |
| DNA | CCGGCGTGCACTCTAAAGTGTGCCAGCACA |
| for | ACGAGGTGCAGCTGGGCAATGAGTGCTGTC |
| UL144B | CTCCTTGTGGCTCTGGACAGCGCGTGACCA |
| (SEQ ID | AAGTGTGCACCGACTACACCAGCGTGACCT |
| NO: 8) | GCACACCCTGTCCTAACGGCACCTATGTGT |
| | CCGGCCTGTACAACTGCACCGATTGCACCC |
| | AGTGCAACGTGACCCAAGTGATGATCCGGA |
| | ACTGCACCAGCACCAACAACACCGTGTGCG |
| | CCCCTAAGAACCACACCTACTTTAGCACCC |
| | CTGGCGTGCAGCACCACAAGCAGAGGCAGC |
| | AGAATCACACAGCCCACATCACCGTGAAGC |
| | AGGGCAAGAGCGGCAGACACACACACCACC |
| | ATCACCATCACTGATAAGCTT |
| Codon | GAATTCCCGCCGCCACCATGGGCTGGTCCT |
| optimized | GCATCATTCTGTTTCTGGTGGCCACAGCCA |
| DNA | CCGGCGTGCACAGCAAGATGTGCAAGCCCG |
| for | ACGAAGTGAAGCTGGGCAACCAGTGCTGTC |
| UL144C | CTCCTTGTGGCAGCGGCCAGAAAGTGACCA |
| (SEQ ID | AAGTGTGCACCGAGAACAGCGGCATCACCT |
| NO: 9) | GTACACTGTGCCCCAACGGCACATACCTGA |
| | CCGGCCTGTACAACTGCACCAATTGCACCC |
| | AGTGCAACGACACCCAGATCACCGTGCGGA |
| | ATTGCACCAGCACCAACAACACCATCTGCG |
| | CCAGCAAGAACCACACCAGCTTTAGCACAC |
| | TGGGCGTGCAGCACCACAAGCAGAGGCAGC |
| | AGAATCACACAGCCCACGTGACAGTGAAGC |
| | AGGGAAAGAGCGGCAGACACACCCACCACC |
| | ACCATCACCATTGATAAGCTT |

We claim:

1. An immunoassay kit for selectively detecting cytomegalovirus in a biological sample, the kit comprising:
(a) a capture reagent comprising a fragment of ULL144 protein, and
(b) a detection reagent.

2. The immunoassay kit of claim 1, wherein the capture reagent comprises a UL144 protein selected from SEQ ID NO: 4-6 or a sequence having about at least 90% sequence similarity to SEQ ID NO:4-6.

3. The immunoassay kit of any claim 1, wherein the capture reagent is attached to a solid or semi-solid support.

4. The immunoassay kit of claim 3, wherein the capture reagent is immobilized on the solid or semisolid support.

5. The immunoassay kit of claim 1, wherein the capture reagent is coated on a microtiter plate.

6. The immunoassay kit of claim 1, wherein the detection agent is a detectable antibody.

7. The immunoassay kit of claim 6, wherein the detectable antibody is a monoclonal antibody.

8. The immunoassay kit of claim 1, wherein the detection agent is biotinylated and the kit further comprises avidin or streptavidin-peroxidase and 3,3′,5,5′-tetramethyl benzidine.

9. The immunoassay kit of claim 1, wherein the kit further comprises reagents for colorimetric detection.

10. The immunoassay of claim 1, wherein the kit further comprises a fluorometric reagent that amplifies the signal of the detection agent in a detection buffer.

11. The immunoassay kit of claim 1, wherein the immunoassay is a multiplex assay capable of detecting two or more CMV serotypes, the immunoassay comprising:
a) a first capture reagent to a first serotype and a second capture reagent to a second serotype, wherein the two capture reagents are in different detection zones in the assay; and
b) the detection agent, wherein the two different detection zones are able to detect the two or more CMV serotypes.

12. The multiplex immunoassay kit of claim 11, wherein the assay further comprises a third capture reagent to a third serotype, wherein the third capture reagent is in a third detection zone.

13. The multiplex immunoassay kit of claim 12, wherein the first, second and third capture reagent are selected from
i) UL144A protein of serotype A or a polypeptide having about at least 90% sequence similarity to UL144A serotype A;
ii) UL144A protein of serotype B or a polypeptide having about at least 90% sequence similarity to UL144A serotype B; and
iii) UL144A protein of serotype C or a polypeptide having about at least 90% sequence similarity to UL144A serotype C.

14. The multiplex immunoassay kit of claim 11, wherein the detection zones may be separate wells on a microtiter plate.

15. The multiplex immunoassay of claim 11, wherein the detection zones may be separate channels in a lateral flow device.

16. The multiplex immunoassay kit of claim 12, wherein the first, second and third capture reagents are selected from:
i) UL144A protein of serotype A of SEQ ID NO:4 or a polypeptide having about at least 90% sequence similarity to UL144A serotype A;
ii) UL144A protein of serotype B of SEQ ID NO:5 or a polypeptide having about at least 90% sequence similarity to UL144A serotype B; and
iii) UL144A protein of serotype C of SEQ ID NO:6 or a polypeptide having about at least 90% sequence similarity to UL144A serotype C.

17. A method of detecting cytomegalovirus infection in a subject, the method comprising:
(a) providing an immunoassay comprising one or more capture reagents specific for CMV serotype A, B or C, preferably wherein the capture reagent is bound to a solid or semi-solid support;
(b) contacting the immunoassay with a biological sample under conditions which allow of CMV antibodies if present in the biological sample to bind to the one or more capture reagents;
(c) adding the detection reagent, and
(d) detecting the complex formed between the capture reagent, CMV antibody and detection reagent within the immunoassay.

18. The method of claim 17, wherein the one or more capture reagents are selected from
i) UL144A protein of serotype A of SEQ ID NO:4 or a polypeptide having at least 90% sequence similarity to UL144A serotype A;
ii) UL144A protein of serotype B of SEQ ID NO:5 or a polypeptide having at least 90% sequence similarity to UL144A serotype B; and
iii) UL144A protein of serotype C of SEQ ID NO:6 or a polypeptide having at least 90% sequence similarity to UL144A serotype C.

19. The method of claim 17, wherein the method differentiates between subjects who have been infected with CMV and subjects who have been vaccinated against CMV.

20. A method for selectively detecting the presence of CMV antibodies in a test sample, comprising the steps of:
a) providing a test sample suspected of containing CMV antibodies;
b) adding a quantity of the polypeptide selected from SEQ ID NO:4-6 to the sample, the quantity being sufficient to produce a detectable level of binding activity by CMV antibodies in the test sample; and
c) detecting the presence of CMV bound to said polypeptide in the test sample by a detection reagent.


SEQUENCE LISTING (11 sequences)

SEQ ID NO: 1 - protein, 167 aa, synthetic construct
MKPLIMLICFAVILLQLGVTKVCQHNEVQLGNECCPPCGSGQRVTKVCTDYTSVTCTPCPNGTYVSGLYNCTDCTQCNVTQVMIRNCTSTNNTVCAPKNHTYFSTPGVQHHKQRQQNHTAHITVKQGKSGRHTLAWLSLFIFLVGIILLILYLIAAYRSERCQQCCS

SEQ ID NO: 2 - protein, 166 aa, synthetic construct
MKPLVMLILLSMLLACIGKTEICKPEEVQLGNQCCPPCKQGYRVTGQCTQYTSTTCTLCPNGTYVSGLYNCTNCTECNDTEVTIRNCTSTNNTVCASKNYTSFSVPGVQHHKQRQNHTAHVTVKQGKSGRHTLAWLSLFIFLVGIILLILYLIAAYRSERCQQCCS

SEQ ID NO: 3 - protein, 167 aa, synthetic construct
MKPLVMLICFGVFLLQLGGSKMCKPDEVKLGNQCCPPCGSGQRVTKVCTENSGITCTLCPNGTYLTGLYNCTNCTQCNDTQITVRNCTSTNNTICASKNHTSFSTLGVQHHKQRQQNHTAHVTVKQGKSGRHTLAWLSLFIFLVGIILLILYLIAAYRSERCQQCCS

SEQ ID NO: 4 - protein, 138 aa, synthetic construct
MGWSCIILFLVATATGVHSKVCQHNEVQLGNECCPPCGSGQRVTKVCTDYTSVTCTPCPNGTYVSGLYNCTDCTQCNVTQVMIRNCTSTNNTVCAPKNHTYFSTPGVQHHKQRQQNHTAHITVKQGKSGRHTHHHHHH

SEQ ID NO: 5 - protein, 139 aa, synthetic construct
MGWSCIILFLVATATGVHSKTEICKPEEVQLGNQCCPPCKQGYRVTGQCTQYTSTTCTLCPNGTYVSGLYNCTNCTECNDTEVTIRNCTSTNNTVCASKNYTSFSVPGVQHHKQRQNHTAHVTVKQGKSGRHTHHHHHH

SEQ ID NO: 6 - protein, 138 aa, synthetic construct
MGWSCIILFLVATATGVHSKMCKPDEVKLGNQCCPPCGSGQRVTKVCTENSGITCTLCPNGTYLTGLYNCTNCTQCNDTQITVRNCTSTNNTICASKNHTSFSTLGVQHHKQRQQNHTAHVTVKQGKSGRHTHHHHHH

SEQ ID NO: 7 - DNA, 444 bp, synthetic construct
  source 1..444: mol_type = other DNA
gaattcccgccgccaccatgggctggtcctgcatcattctgtttctggtggccacagccaccggcgtgcacagcaaaacagagatctgcaagcccgaggaagtgcagctgggcaatcagtgctgtcctccttgcaagcagggctacagagtgaccggccagtgtacccagtacaccagcaccacctgtacactgtgccccaacggcacctatgtgtccggcctgtacaactgcaccaattgcaccgagtgcaacgacaccgaagtgaccatccggaactgcacctccaccaacaataccgtgtgcgccagcaagaactacaccagcttttccgtgcctggcgtgcagcaccacaagcagagacagaatcacacagcccacgtgaccgtgaagcagggcaaaagcggcagacacacccaccaccaccatcaccattgataagctt

SEQ ID NO: 8 - DNA, 441 bp, synthetic construct
  source 1..441: mol_type = other DNA
gaattcccgccgccaccatgggctggtcctgcatcattctgtttctggtggccacagccaccggcgtgcactctaaagtgtgccagcacaacgaggtgcagctgggcaatgagtgctgtcctccttgtggctctggacagcgcgtgaccaaagtgtgcaccgactacaccagcgtgacctgcacaccctgtcctaacggcacctatgtgtccggcctgtacaactgcaccgattgcacccagtgcaacgtgacccaagtgatgatccggaactgcaccagcaccaacaacaccgtgtgcgcccctaagaaccacacctactttagcacccctggcgtgcagcaccacaagcagaggcagcagaatcacacagcccacatcaccgtgaagcagggcaagagcggcagacacacacaccaccatcaccatcactgataagctt

SEQ ID NO: 9 - DNA, 441 bp, synthetic construct
  source 1..441: mol_type = other DNA
gaattcccgccgccaccatgggctggtcctgcatcattctgtttctggtggccacagccaccggcgtgcacagcaagatgtgcaagcccgacgaagtgaagctgggcaaccagtgctgtcctccttgtggcagcggccagaaagtgaccaaagtgtgcaccgagaacagcggcatcacctgtacactgtgccccaacggcacatacctgaccggcctgtacaactgcaccaattgcacccagtgcaacgacacccagatcaccgtgcggaattgcaccagcaccaacaacaccatctgcgccagcaagaaccacaccagctttagcacactgggcgtgcagcaccacaagcagaggcagcagaatcacacagcccacgtgacagtgaagcagggaaagagcggcagacacacccaccaccaccatcaccattgataagctt

SEQ ID NO: 10 - protein, 162 aa, synthetic construct
MKPLVMLILLSILLACIGKTECKPEEVQLGNQCCPPCKQGYRVTGQCTQYTSTTCTLCPNGTYVSGLYNCTNCTECBDTEVTURBCTSTNNTVCASJNHTSKSVOGVQHHJQRQNHTAHVTVKSGRHTLAWLSLFIFLVGIILLILYLIAAYRSERCQQCCS

SEQ ID NO: 11 - protein, 164 aa, synthetic construct
LVMLICFAVILLQLGVTKVCQHNEVQLGNECCPPCGSGQRVTKVCTDYTSVTCTPCPNGTYVSGLYNCTDCTQCNVVTQVMIRNCTSTNNTVCASKNYTSFSISGVQHHKQRQNHTAHVTVKQGKSGRHTLAWLSLFIFLVGIILLILYLIAAYRSEKCQQCCS